(19) United States
(12) Patent Application Publication
(10) Pub. No.: US 2004/0009210 A1
(43) Pub. Date: Jan. 15, 2004

(54) Wound management products incorporating cationic compounds

(75) Inventors: David W. Koenig, Menasha, WI (US); David J. Tyrrell, Appleton, WI (US); Mike T. Goulet, Neenah, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/330,460

(22) Filed: Dec. 26, 2002

Related U.S. Application Data
(60) Provisional application No. 60/394,634, filed on Jul. 9, 2002.

(51) Int. Cl. A61K 9/70

(52) U.S. Cl. 424/445

(57) ABSTRACT

Novel wound management products are disclosed. The products incorporate an increased amount of cationic compounds in the fibrous matrix of the substrate which allow the product to bind and remove various microbes from the surface of a wound. The products disclosed herein do not necessarily kill microbes on the wound's surface, but dislodge and bind the microbe through electrostatic interactions between the product and the microbe and allow the microbe to be removed by a positively charged substrate.

### CROSS-REFERENCE TO RELATED APPLICATIONS

This application claims priority from U.S. Provisional Patent Application No. 60/394,634 filed on Jul. 9, 2002, the entirety of which is hereby incorporated by reference.

### BACKGROUND OF THE INVENTION

The present invention relates to wound dressing products useful in removing microbes from acute and chronic wounds. More particularly, the present invention relates to wound dressing products which are highly effective in binding and removing from wounds, wound discharges, and surrounding skin, a broad range of harmful microorganisms. The wound dressing products of the present invention, which may include gauzes, wipes, wraps, pads, bandages, etc., are useful in the treatment of both chronic and acute wounds, and incorporate increased concentrations of cationic compounds, such as, for example, octadecyldimethyltrimethoxylsilpropylammonium chloride, which have an effective charge density of from about 0.1 microequivalents/g to about 8000 microequivalents/g, or more which electrically alter the fibers comprising the product. When the fibers of the wound dressing impregnated with the cationic compound contact the wound, surrounding skin, and/or wound discharge, the cationic compound contained on and/or within the dressing binds microbes onto the cationic particles and dressing such that the microbe may be removed from the wound, surrounding skin, and/or discharge. This provides a significant advantage in that the microbe material is not simply dislodged from the wound surface, but is dislodged and bound simultaneously and, therefore, is no longer available for dispersion and future contamination of the wound or surrounding areas.

The outer layer of skin surrounding the body performs numerous functions including an important protective function as a barrier against infection, as well as serving as a means of regulating the exchange of heat, fluid, and gas between the body and the external environment. When skin is removed or damaged by being abraded, burned or lacerated, the protective function is seriously diminished. Areas of damaged skin have been conventionally protected by the application of a wound dressing, such as a wrap or bandage, which helps facilitate wound healing by acting as a skin substitute and protective barrier.

Wounds to the skin and the underlying tissue may be caused by an external insult such as friction, abrasion, laceration, burning or chemical irritation. Damage to such tissues may also result from internal metabolic or physical dysfunction, including but not limited to bone protrudence, diabetes, circulatory insufficiencies, or inflammatory processes. Normally, tissue damage initiates the physiological processes of regeneration and repair. In broad terms, this regeneration and repair process is commonly referred to as the wound healing process.

The wound healing process usually progresses through distinct stages leading to the eventual closure, and restoration of the natural function of the tissues. Injury to the skin initiates an immediate vascular response characterized by a transient period of vasoconstriction, followed by a more prolonged period of vasodilation. Blood components infiltrate the wound site, endothelial cells are released, exposing fibrillar collagen, and platelets attach to the exposed sites. As platelets become activated, components are released which initiate events of the intrinsic coagulation pathway. At the same time, a complex series of events trigger the inflammatory pathways generating soluble mediators to direct subsequent stages of the healing process.

Normally, the wound healing process is uneventful and may occur regardless of any intervention, even in the case of acute or traumatic wounds. However, where an underlying metabolic condition or perpetual insult such as pressure is a contributing factor, the natural wound healing process may be retarded or completely arrested, resulting in a chronic wound. Trends in modern medical practices have shown that the wound healing of both acute and chronic wounds may be significantly improved by clinical intervention using methods and materials that significantly reduce or eliminate the existence of microbes on the wound surface that may cause further complications such as infection.

Infection control of both chronic and acute wounds has, to date, primarily focused on chemical agents which lyse or inhibit the growth of microorganisms present on and below the wound surface and/or in fluids discharged by the wound during healing. Although potentially effective in reducing the amount of microorganisms present on the wound surface, such bactericidal and bacteriostatic products have the potential to produce resistant strains which may lead to serious, or even fatal, outcomes for the host. Further, residue from bacteria cells not removed from the wound surface can lead to further infection. As such, a need exists in the art for an improved wound dressing or wound treatment product capable of removing microbes from acute and chronic wounds which does not suffer from the same shortcomings as described above.

### SUMMARY OF THE INVENTION

The present invention provides wound dressing and management products which can bind and remove various microbes from wounds on the skin, as well as from skin surrounding a wound and from discharged liquids from wounds. The wound dressing and management products of the present invention are highly effective in dislodging and binding numerous microbes, such as Candida albicans, from the surface of the wound. Significantly, the products of the present invention do not necessarily kill microbes on the wound's surface or in the immediate area of the wound during removal, but dislodge and bind the microbes through electrostatic interactions between the product and the microbe. It has been discovered that by providing a wound dressing product comprising a sufficient amount of cationic compounds having an effective charge density of from about 0.1 microequivalents/g to about 8000 microequivalents/g or more, the fibers comprising the product can be electrically altered such that the resulting product has a Positive Charge Index as defined herein of at least about 52. Such a Positive Charge Index allows numerous types of microbes to be electrostatically dislodged from the wound surface, captured and carried away. The cationic compound-containing wound management products of the present invention are safe for use around wounds in the skin as microbes are removed from the wound surface without a substantial risk of rupturing, and thus the risk of introduction of byproducts from the microbe into wounds is minimized or eliminated.

Briefly, therefore, the present invention is directed to a wound management product. The product comprises a substrate carrying a cationic compound capable of binding microbes located on the surface of a wound. The cationic compound has an effective charge density of from about 0.1 microequivalents/g to about 8000 microequivalents/g and a positive charge which imparts a Positive Charge Index of at least about 52 to said product.

The present invention is further directed to a wound management product. The product comprises a substrate carrying a cationic compound capable of binding microbes located on the surface of a wound. The cationic compound has an effective charge density of from about 500 microequivalents/g to about 8000 microequivalents/g and a positive charge which imparts a Positive Charge Index of at least about 52 to said product.

The present invention is further directed to a wound management product. The product comprises a substrate carrying a cationic compound capable of binding microbes located on the surface of a wound. The cationic compound has an effective charge density of from about 1000 microequivalents/g to about 8000 microequivalents/g and a positive charge which imparts a Positive Charge Index of at least about 52 to said product.

The present invention is further directed to a wound management product comprising a woven web material and a cationic compound capable of binding microbes located on the surface of a wound. The cationic compound has an effective charge density of from about 1000 microequivalents/g to about 8000 microequivalents/g and the product has a Positive Charge Index of at least about 52.

The present invention is further directed to a wound management product comprising a non-woven web material and a cationic compound capable of binding microbes located on the surface of a wound. The cationic compound has an effective charge density of from about 1000 microequivalents/g to about 8000 microequivalents/g and the product has a Positive Charge Index of at least about 52.

Other objects and features of this invention will be in part apparent and in part pointed out hereinafter.

### DEFINITIONS

Within the context of this specification, each term or phrase below will include, but not be limited to, the following meaning or meanings:

(a) “Bonded” refers to the joining, adhering, connecting, attaching, or the like, of two elements. Two elements will be considered to be bonded together when they are bonded directly to one another or indirectly to one another, such as when each is directly bonded to intermediate elements.

(b) “Film” refers to a thermoplastic film made using a film extrusion and/or foaming process, such as a cast film or blown film extrusion process. The term includes apertured films, slit films, and other porous films which constitute liquid transfer films, as well as films which do not transfer liquid.

(c) “Layer” when used in the singular can have the dual meaning of a single element or a plurality of elements.

(d) “Meltblown” refers to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface. Meltblown fibers used in the present invention are preferably substantially continuous in length.

(e) “Nonwoven” refers to materials and webs of material which are formed without the aid of a textile weaving or knitting process.

(f) “Polymeric” includes, but is not limited to, homopolymers, copolymers, such as for example, block, graft, random and alternating copolymers, terpolymers, etc. and blends and modifications thereof. Furthermore, unless otherwise specifically limited, the term “polymeric” shall include all possible geometrical configurations of the material. These configurations include, but are not limited to, isotactic, syndiotactic and atactic symmetries.

(g) “Positive Charge Index” refers to the amount of positive charge contained on the surface of a substrate as measured by a Positive Charge Index Assay.

(h) “Positive Charge Index Assay” refers to an eosinol assay which utilizes Eosin Y or Eosin B as a biological stain to measure the Positive Charge Index of a substrate.

(i) “Thermoplastic” describes a material that softens when exposed to heat and which substantially returns to a non-softened condition when cooled to room temperature.

### DETAILED DESCRIPTION OF THE PREFERRED EMBODIMENTS

In accordance with the present invention, it has been discovered that numerous microbes such as, for example, Candida albicans, attached to the surface of a wound or contained in the discharge of a wound, can be effectively dislodged, captured and removed away from the wound's surface through the use of a wound management product carrying a suitable amount of cationic compounds, such as, for example, octadecyldimethyltrimethoxylsilpropylammonium chloride, having a suitable effective charge density or anion exchange capacity which modifies the overall charge density of the product. Surprisingly, the wound management products of the present invention are highly effective in removing microbes from a wound and surrounding area, yet are very gentle and non-irritating to the wound. Advantageously, the wound management products of the present invention do not necessarily kill cells or puncture cell walls during skin cleansing, but simply dislodge and bind the contaminant from the wound surface allowing its removal. By facilitating the release and binding of microbes located in and around a wound, the products of the present invention significantly improve wound healing and skin health without being substantially irritating to the wound and/or surrounding skin.

The cationic compounds described herein can be incorporated into or onto a substrate, such that the substrate carries the cationic compound, utilizing numerous methods. In one embodiment of the present invention, the cationic compounds are impregnated into the fibers comprising the underlying substrate of the wound product during the substrate manufacturing process. Although generally referred to herein as “pulp fibers” or “cellulose fibers,” it should be recognized that various types of fibers, including wood pulp fibers and synthetic and polymer-type fibers, are suitable for substrate use in the wound management products of the present invention, and are within the scope of the present invention. Suitable substrates for incorporation of the cationic compounds include, for example, cellulosic materials, coform materials, woven webs, non-woven webs, spunbonded fabrics, meltblown fabrics, knit fabrics, wet laid fabrics, needle punched webs, or combinations thereof. Particularly preferred substrates are woven and non-woven webs.

The cationic compounds contained in the wound management products of the present invention appear to electrostatically interact with microbes and other contaminants located in and around wounds. Through this interaction, the products are able to dislodge the microbe and/or contaminant from the surface of the wound and bind the microbe and/or contaminant onto the wound management product such that it may be carried away from the wound. As used herein, the term “microbe” should be read to include bacteria, yeast and viruses. It is believed that the cationic compounds located on or in the fibers of the wound management product interact with the overall net negative charge associated with microorganisms and other contaminants, thereby binding the microorganism and/or contaminant and removing it from the wound surface. The wound management products of the present invention are also useful in removing microbes and contaminants from the fluid produced by a wound during the healing process.

The cationic compounds impregnated into or onto the wound management products of the present invention do not necessarily kill or inhibit the growth of microbes, but displace and bind the predominantly negatively charged microbes or other contaminants from the wound surface through positive-negative or negative-positive electrostatic interactions. This is highly advantageous in that the wound management products of the present invention do not require an antimicrobial, bactericidal or bacteriostatic ingredient to be highly effective in safely cleaning wounds and surrounding skin. When the wound management products of the present invention are utilized in or around skin wounds, microbes are not simply punctured, killed and left in the wound, but are actually bound to the cationic compounds in or on the fibers of the product and removed from the skin. This may significantly reduce the chance of further infection in and around the wound. Further, the cationic compounds used in the products of the present invention are substantially non-toxic and non-irritating to the wound and surrounding skin.

Without being bound to a particular theory, it appears that by increasing the attractive forces between the wound management product containing the cationic compounds and the microbe and/or contaminant on or near the wound's surface in excess of the forces attracting the microbe and/or contaminant to the skin, cleaning of the wound and surrounding skin can be significantly enhanced by dislodging and binding the contaminant to the cationic species added to the product. It appears that the cationic compounds interact with the overall net negative charge of the microbe and/or contaminant causing the detachment of the microbe and/or contaminant from the wound through an electrostatic interaction. The interaction between the cationic compounds and the microbe and/or contaminant appears to be stronger than the combined forces of adhesion that retain the microbe and/or contaminant on or near the wound including hydrophobic interactions, electrostatic interactions, and ligand interactions. Because the microbe and/or contaminant is released from the wound and bound to the charge modified wound management product, it may be easily and efficiently carried away by the product. This is highly advantageous over more traditional cleaning products as the contaminant is not merely dislodged from the wound surface, but is dislodged and then removed from the wound's surface through interactions with the substrate containing the cationic compounds. A suitable amount of cationic compounds are added to the wound management products of the present invention such that the forces binding the contaminant to the skin surface, such as hydrophobic interactions, electrostatic interactions, and ligand interactions, can be overcome by the attraction to the cationic species.

An important novel aspect of the present invention is that the charge-modified wound management products of the present invention significantly improve wound cleanliness and health without necessarily killing microorganisms present on the surface of the wound. As mentioned above, this can be a critical factor when products are utilized around wounds. Typically, when microorganisms are killed by antimicrobial or bactericidal agents, which are common in some wound management products, the outer wall of the microorganism is penetrated and opened to allow access by a killing agent such as, for example, an organic acid. Although this typically results in a kill of the microorganism, the inside contents of the microorganism can “spill out” into an open wound and lead to further complications or increased infections. This significant problem is minimized or eliminated by the present invention which releases the microorganism from the wound surface such that it can be transferred to a substrate surface and carried away. The interaction between microorganisms or other contaminants and the charge altered wound management products of the present invention results in an actual energy transfer, i.e., energy is released and recaptured in the dislodging and rebinding of contaminants from the wound surface to the cleaning substrate. This cleaning mechanism may also be important for the control of certain other skin problems, such as diaper rash.

The cationic compounds of the present invention utilized to increase the overall cationic charge of a product can be easily incorporated into various wound management products such as wraps, dressings, bandages, pads, wipes, gauze pads and gauze wraps, etc., during the manufacturing process. During the manufacture of various wound management products, physical and/or optical properties of the product are often altered by the addition of chemical additives. Generally, chemical additives such as softeners, colorants, brighteners, and strength agents are added to the fiber slurry upstream of the headbox in a paper making machine during the manufacturing or converting stages of production to impart certain attributes to the finished product. These chemical additives are typically mixed in a stock chest or stock line where the fiber slurry has a fiber consistency of from about 0.015 to about 5 percent.

To improve the adsorption of wet end chemical additives, the chemical additives are often modified with functional groups to impart an electrical charge when in water. The electrokinetic attraction between positively charged chemical additives and the anionically charged fiber surfaces aids in the deposition and retention of the chemical additives onto the fibers. The amount of the chemical additive that can ultimately be adsorbed or retained in the paper machine wet end generally follows an adsorption curve exhibiting diminishing incremental adsorption with increasing concentration. As a result, the adsorption of water soluble or water dispersible chemical additives may be significantly less than 100 percent, particularly when trying to achieve high chemical additive loading levels.

In the alternative, the chemical additives mentioned above may be applied onto pulp fiber surfaces in the initial or primary pulp processing, providing more consistent chemical additive additions to the pulp fiber and a reduction or elimination of unretained chemical additives in the process water on a paper machine. With this method, the chemical treatment of the pulp fibers may occur prior to, during, or after the drying phase of the pulp processing. The generally accepted methods of drying include flash drying, can drying, flack drying, through air drying, infrared drying, fluidized bed drying, or any method known in the art. The addition of cationic compounds to increase the overall cationic charge of the finished product in accordance with the present invention may also be applied to wet lap pulp processes without the use of dryers.

The method for applying the cationic additives of the present invention to the pulp fibers may be used in a wide variety of pulp finishing processing, including dry lap pulp, wet lap pulp, crumb pulp, and flash dried pulp operations. By way of illustration, various pulp finishing processes (also referred to as pulp processing) are disclosed in Pulp and Paper Manufacturing: The Pulping of Wood, 2nd Ed., Volume 1, Chapter 12 (Ronald G. MacDonald, Editor), which is incorporated by reference. Various methods may be used to apply the cationic compounds described herein to achieve the desired Positive Charge Index including, but not limited to, direct addition to a fiber slurry, spraying, coating, foaming, printing, size pressing, or any other method known in the art. Further, in situations where additional chemical additives other than the cationic compounds of the present invention are to be employed, the chemical additives may be added to the fibrous web in sequence to reduce interactions between the chemical additives.

Typically, bleached-chemical virgin pulp fiber used in the manufacture of wound management products has a low initial Positive Charge Index when introduced into the manufacturing process, and hence has an overall negative charge. Other types of virgin pulp fiber, such as unbleached-chemical fiber, which may have an even lower initial Positive Charge Index may also be used in accordance with the present invention, but are typically less preferred. As discussed above, during processing numerous chemical additives, most of which are cationic in nature, such as softeners, are added to improve the overall characteristics of the finished product. The total addition of cationic compounds to pulp during the conventional manufacturing of wound management products may typically result in a slightly cationically charged finished product. Such a conventional finished product may have a Positive Charge Index of no more than about 50.

In accordance with the present invention, an amount of cationic compounds in excess of the amounts typically used in the manufacturing process of wound management products is added to the pulp during or after manufacturing to alter the electric charge of the cellulose fibers comprising the product from negative to positive (or from very slightly positive to more positive) to increase the Positive Charge Index of the finished wound management product such that the product retains a strongly positive surface charge. Such a surface charge makes the wound management product highly effective in binding and removing contaminants from the skin's surface through electrostatic interactions.

As noted above, the Positive Charge Index of a wound management product is measured in accordance with the present invention utilizing a Positive Charge Index Assay. The Positive Charge Index Assay can utilize Eosin Y or Eosin B as noted below as the reagent. The Positive Charge Index Assay is set forth below.

Positive Charge Index Assay For Determining the Positive Charge Index of a Substrate

The amount of positive charge imparted onto a substrate or product, such as a base sheet or woven or non-woven web, for example, can be measured in accordance with the present invention using the Positive Charge Index Assay including an anionic dye binding assay. The Positive Charge Index Assay utilizes the dye Eosin Y, which is a biological stain for alkaline materials. Eosin B can optionally be utilized in place of Eosin Y. The Positive Charge Index Assay is carried out as follows:

Step 1: Cut the substrate to be evaluated into two squares approximately 2 centimeters by 2 centimeters. The first square will be stained with Eosin Y as described herein and optically evaluated. The second square will be subjected to the same Eosin Y staining procedure described herein with the exception that the second square will not be stained with Eosin Y; that is, the second square will undergo each and every step as the first square, except Steps 5 and 6 below.

Step 2: Introduce filter paper, such a Whatman #4 Qualitative 125 millimeter filter paper or equivalent, into a Buchner Funnel attached to a vacuum source.

Step 3: Start the vacuum, and wash the filter paper with deionized water.

Step 4: Allow the filter paper to dry.

Step 5: Place the test substrate on top of the dry filter paper and saturate the substrate with 0.75 milliliters of 0.5% (weight/volume) Eosin Y prepared in deionized water.

Step 6: Allow the test substrate to soak in the Eosin Y for 2 minutes and then cover the test substrate with a dry piece of filter paper.

Step 7: Wash the test substrate through the filter paper for 3 minutes with deionized water.

Step 8: Remove the test substrate with forcepts and place it on a dry piece of filter paper and allow it to dry completely.

Step 9: Measure CIELAB Color Space of the dried test substrate using a Minolta CM-508d Spectrophotometer, or similar equipment. The spectrophotometer is set for CIELAB Color Space with the following parameters: Target Status CREEMM, Color Mode L*a*b*, Observer 10°, and the primary Illuminant D65. A standard white block supplied by the spectrophotometer manufacturer is utilized for calibration of the instrument.

Step 10: Calculate the DE*ab value of the Eosin Y stained test substrate using an un-stained test substrate for comparison. The DE*ab value is equal to the Positive Charge Index. The higher the Positive Charge Index, the higher the positive charge on the substrate. The CIE Color System Values are set forth below:

L*=Lightness=A value 0 to 100

a*=Color coordinate red-verses-green

b*=Color coordinate yellow-verses-blue

C=Chroma=[(a*)²+(b*)²]^{1/2}

h=Hue angle=arctan(b*/a*)

E=Color difference=[(L*)²+(a*)²+(b*)²]^{1/2}

DL*=L*_{Eosin Stained Substrate}−L*_{Unstained Substrate}

Da*=a*_{Eosin Stained Substrate}−a*_{Unstained Substrate}

Db*=b*_{Eosin Stained Substrate}−b*_{Unstained Substrate}

DE*ab=[(DL*)²+(Da*)²+(Db*)²]^{1/2}

The cationic compounds useful in the present invention to increase the overall effective cationic charge density of a finished product can easily be incorporated into various wound management products. As used herein, the term “cationic compound” means any compound or ingredient which increases the overall cationic charge of the fibers comprising a wound management product when the fibers are wetted. Preferably, the cationic compounds used in accordance with the present invention to increase the overall effective charge density of a finished product are non-antagonistic to pulp fibers or to other additives utilized in the manufacturing process. Further, it is preferred that the additional cationic compounds added to the pulp in accordance with the present invention do not substantially adversely affect the overall strength and integrity of the resulting modified product.

Examples of suitable cationic compounds that can be utilized to increase the overall effective cationic charge density of the wound management products of the present invention include, for example, polyquaternary ammonium compounds, such as those sold under the tradename Bufloc 535 (Buckman Laboratories International, Memphis, Tenn.), Nalco 7607 (ONDEO NALCO Company, Naperville, Ill.), Reten 201 (Hercules Inc., Wilmington, Del.), Cypro 515 (CIBA Speciality Chemicals, Suffolk, Va.), Bufloc 5554 (Buckman Laboratories International, Memphis, Tenn.), and Busperse 5030 (Buckman Laboratories International, Memphis, Tenn.) and cationic polymers, inorganic cationic species, biological cationic polymers, modified chitosan, octadecyldimethyltrimethoxylsilpropylammonium chloride, octadecyldimethoxylsilpropylammonium chloride, polyacrylamides, diallydimethylammonium chloride, dicyandiamideformaldehyde, epichlorohydrinamine, cationic liposomes, modified starch, 1-methyl-2-Noroleyl-3-oleyl-amidoethyl imidazoline methylsulfate, 1-ethyl-2-Noroleyl-3-oleyl-amidoethyl imidazoline ethylsulfate, trimethylsilylmodimethicone, amodimethicone, polyquaternium-2, polyquaternium-4, polyquaternium-5, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-32, polyquaternium-33, polyquaternium-34, polyquaternium-35, polyquaternium-36, polyquaternium-37, polyquaternium-39, polysilicone-1, polysilicone-2, and mixtures and combinations thereof. Especially preferred compounds include quaternary compounds, polyelectrolytes, octadecyldimethoxylsilpropylammonium chloride, 1-methyl-2-Noroleyl-3-oleyl-amidoethyl imidazoline methylsulfate, and 1-ethyl-2-Noroleyl-3-oleyl-amidoethyl imidazoline ethylsulfate. It would be recognized by one skilled in the art that other cationic compounds commonly used in pulp manufacturing processes could also be utilized in accordance with the present invention to significantly increase the overall cationic effective charge density of the resulting product.

The cationic compounds for incorporation into the wound management products of the present invention have a net cationic charge, and may sometimes be referred to as anion exchangers. Typically, the products of the present invention contain cationic compounds having sufficient positive charge to impart improved cleaning characteristics into the products through electrostatic interactions with microbes and/or contaminants and skin. The amount of “cationic charge” on a particular compound can vary substantially and can be measured utilizing several different units. Anionic exchangers are sometimes referred to as having a “capacity” which may be measured in microequivalents per gram or milliequivalents per gram, or may be measured in terms of the amount of a certain compound or protein that the anionic exchanger will bind. Still another way of referring to the amount of positive charge is in terms of micro or milliequivalents per unit area. One skilled in the art will recognize that the exchange capacity units can be converted from one form to another to calculate proper amounts of anion exchanger for use in the present invention.

In accordance with the present invention, the chemical additives utilized to increase the overall effective cationic charge density of the resulting product have a cationic charge. Cationic compounds useful in the present invention typically have an effective charge density of from about 0.1 microequivalents/g to about 8000 microequivalents/g, more preferably from about 100 microequivalents/g to about 8000 microequivalents/g, still more preferably from about 500 microequivalents/g to about 8000 microequivalents/g, and most preferably from about 1000 microequivalents/g to about 8000 microequivalents/g. Although effective charge densities of more than about 8000 microequivalents/g can be used in the wound management products of the present invention, such a large charge density is not typically required to realize the benefit of the present invention, and may result in the deterioration of product properties. As the effective charge density of the cationic material increases, the amount of cationic material required to be added to the pulp manufacturing process typically decreases. Generally, from about 0.01% (by weight of the substrate) to about 25% (by weight of the substrate), preferably from about 0.01% (by weight of the substrate) to about 10% (by weight of the substrate) of cationic material having the above-described effective charge density will be sufficient to increase the overall cationic charge of the resulting product sufficiently for purposes of the present invention. The actual amount of cationic material required for introduction into the pulp manufacturing process may be influenced by numerous other factors including, for example, the amount of steric hindrance in the pulp fibers due to other additives present in the pulp fiber environment, the accessibility of the charges on the pulp fibers, competitive reactions by cationic materials for anionic sites, the potential for multilayer adsorption into the pulp fiber, and the potential for precipitation of anionic materials out of solution.

Without being bound to a particular theory, it is believed that many of the cationic molecules (which may sometimes also be referred to as “softeners” or “debonders”) suitable for use in accordance with the present invention have a cationic charge by virtue of a quaternary nitrogen moiety. During the manufacturing of the wound management product, this cationic charge may be used to attract the cationic molecule to the fiber surface, which is typically anionic in nature. The cationic compounds suitable for use in the present invention may have hydrophobic side chains which impart hydrophobicity to the molecule, making these molecules substantially non-water soluble. As such, these cationic compounds are believed to actually exist in solution as micelles of cationic compound molecules, where the hydrophobic tails are in the interior of the micelle and the cationic charges are exposed to the water phase. When a micelle cluster is adsorbed onto the fiber, more than one molecule is present on the surface, thus creating a site on the fiber with an excess of cationic charge. Once dried, these cationic molecules are likely associated with a counterion (although it may be possible that some are present without counterions which may create a static cationic charge) to form a net neutral charge. When the treated substrate comes into contact with an aqueous media such as the moisture associated with a wound, the counterion is free to dissociate and thus leaves the fiber cationically charged in the region with adsorbed cationic molecules. The cationic charge on the surface of the substrate is then able to attract and retain various microbes which typically have a negatively charged outer surface.

In one embodiment of the present invention, the cationic compounds of the present invention can be incorporated into a substrate which can be a woven web, non-woven web, spunbonded fabric, meltblown fabric, knit fabric, wet laid fabric, needle punched web, cellulosic material or web, and combinations thereof, for example, to create wound management products such as bandages, gauzes, wraps, wipes, dressings, and the like.

The addition of the cationic compounds to the substrate may be performed using a liquid application treater such as a DAHLGREN® LAS. This application system applies a wet solution comprising the cationic compounds to the substrate followed by a drying process to produce a dry substrate containing the cationic compounds. This system is commercially available and well known to those skilled in the art.

The wound management products described herein having an increased effective cationic charge density are highly effective in binding and removing microbes and/or certain contaminants from a wound's surface and surrounding skin. Although not required, the products described herein can be used in combination with other additives to further increase the efficacy of the product under certain circumstances. For example, the products described herein can be used in combination with antimicrobial agents, detergents, microbiocides, colorants, or other additives or skin sensitizing chemicals.

### EXAMPLE 1

In Part 1 of this Example, several bath tissues including both commercially available bath tissues, and modified bath tissues, along with other non-cellulosic cleaning sheets were evaluated to determine their Positive Charge Index utilizing a Positive Charge Index Assay. In Part 2 of this Example, three of the tested bath tissues and two cleaning sheets were evaluated for their efficacy in removing Candida albicans from a surface.

Part 1:

The following samples (test substrates) were evaluated to determine their Positive Charge Index: (1) Scott® commercial bath tissue; (2) Charmin® commercial bath tissue; (3) Northern® commercial bath tissue; (4) Cottonelle® bath tissue without any cationic softener added; (5) Cottonelle® commercial bath tissue (9.4 kilogram/metric ton of cationic softener added); (6) Cottonelle® bath tissue with 15 kilogram/metric ton of cationic softener added; (7) DSX basesheet without the addition of any SILGARD (octadecyldimethyltrimethoxylsilpropyl-ammonium chloride); and (8) DSX basesheet with the addition of 0.7% (by weight) SILGARD.

The cationic softener added to the Cottonelle® bath tissues (test substrate numbers 5 and 6) was 1-methyl-2-Noroleyl-3-oleyl amidoethyl imidazolinium methosulfate obtained from Goldschmidt Ag (Hopewell, Va.) which has an effective charge density (measured) of about 1300 microequivalents/gram. The cationic softener levels referred to above (9.4 kilogram/metric ton and 15 kilogram/metric ton) are the cationic softener additive loadings based on the outer layer furnish in the sheet. The outer two layers (of a three layer sheet) made up about 65% of the total sheet weight. The remaining about 35% by weight in the center of the product had no cationic softener added to it. As such, the levels of cationic softener based on the total sheet weight are 9.4×0.65, or 6.1 kilogram/metric ton and 15×0.65, or 9.75 kilogram/metric ton based on the total weight of the three layer sheet. The DSX basesheet was a non-cellulosic, non-woven hydroentangled polyester cleaning basesheet.

The Positive Charge Index Assay was done as follows:

Step 1: Each test substrate was cut into two approximate 2 centimeter by 2 centimeter squares. One of the two squares was evaluated optically without any Eosin Y staining, and the other was subjected to the Eosin Y staining as described herein prior to optical evaluation. The test substrate not subjected to the Eosin Y staining was still subjected to each and every other Step of the procedure.

Step 2: A Whatman #4 Qualitative 125 millimeter filter paper (Whatman, Maidstone, England) was introduced into a Buchner Funnel attached to a vacuum source.

Step 3: The vacuum source was activated and the filter paper was washed with deionized water. After washing, the vacuum source was turned off and the filter paper allowed to air dry.

Step 4: The test substrate was introduced onto the dried filter paper in the Buchner Funnel and saturated with 0.75 milliliters of 0.5% (w/v) Eosin Y (Sigma Chemical Company, St. Louis Mo.) prepared in deionized water and allowed to soak for 2 minutes.

Step 5: After soaking, the test substrate was covered with a dry Whatman #4 Qualitative 125 millimeter filter paper and the test substrate washed through the filter paper with deionized water for 3 minutes.

Step 6: After washing, the test substrate was removed with forceps and placed on dry Whatman #4 Qualitative 125 millimeter filter paper and allowed to air dry for approximately 15 minutes.

Step 7: After drying, the CIELAB Color Space of the test substrate was measured using a Minolta CM-508d Spectrophotometer (Minolta, Japan). Standard White (Minolta, Japan) was utilized as the calibration color. The spectrophotometer was set for CIELAB Color Space with the following parameters: Target Status CREEMM, Color Mode L*a*b*, Observer 10°, and the primary illuminant D65.

Step 8: The DE*ab value of the Eosin Y stained test substrate was calculated utilizing the corresponding un-stained test substrate for comparison. The DE*ab value was equal to the Positive Charge Index for the substrate. The higher the Positive Charge Index, the higher the charge on the surface of the substrate. The following CIE Color System Values were utilized:

L*=Lightness=A value 0 to 100

a*=Color coordinate red-verses-green

b*=Color coordinate yellow-verses-blue

C=Chroma=[(a*)²+(b*)²]^{1/2}

h=Hue angle=arctan (b*/a*)

E=Color difference=[(L*)²+(a*)²+(b*)²]^{1/2}

DL*=L*_{Eosin Stained Substrate}−L*_{Unstained Substrate}

Da*=a*_{Eosin Stained Substrate}−a*_{Unstained Substrate}

Db*=b*_{Eosin Stained Substrate}−b*_{Unstained Substrate}

DE*ab=[(DL*)²+(Da*)²+(Db*)²]^{1/2}

The raw data collected on the test substrates is set forth in Table 1, and calculated values are set forth in Table 2.

TABLE 1
CIELAB Color Space Raw Data

| Sample | L* | a* | b* | C | h |
|---|---|---|---|---|---|
| Standard White | 96.003 | 0.004 | −0.008 | 0.009 | 295.282 |
| Unstained Substrate | | | | | |
| Scott ® Commercial | 97.603 | −0.056 | 0.579 | 0.582 | 95.541 |
| Charmin ® Commercial | 97.517 | −0.253 | 1.362 | 1.368 | 100.516 |
| Northern ® | 96.962 | −0.346 | 2.088 | 2.117 | 99.411 |
| Commercial | | | | | |
| Cottonelle ® | 97.399 | −0.120 | 0.897 | 0.905 | 97.608 |
| (0 kg/mt | | | | | |
| cationic softener*) | | | | | |
| Cottonelle ® | 97.529 | −0.157 | 1.021 | 1.033 | 98.740 |
| Commercial | | | | | |
| (9.4 kg/mt | | | | | |
| catiomic softener) | | | | | |
| Cottonelle ® | 97.433 | −0.164 | 1.042 | 1.055 | 98.952 |
| (15 kg/mt | | | | | |
| cationic softener) | | | | | |
| DSX Basesheet | 97.020 | 0.019 | 0.284 | 0.285 | 86.128 |
| (0% SILGARD) | | | | | |
| DSX Basesheet | 97.502 | 0.546 | −3.549 | 3.591 | 278.747 |
| (0.7% SILGARD) | | | | | |
| Eosin Y Stained | | | | | |
| Substrate | | | | | |
| Scott ® | 89.491 | 20.500 | −6.179 | 21.411 | 343.228 |
| Commercial | | | | | |
| Charmin ® | 78.279 | 41.203 | −12.813 | 43.150 | 342.725 |
| Commercial | | | | | |
| Northern ® | 83.664 | 32.199 | −8.910 | 33.409 | 344.532 |
| Commercial | | | | | |
| Cottonelle ® | 89.708 | 17.303 | −5.429 | 18.135 | 342.580 |
| (0 kg/mt | | | | | |
| cationic softener) | | | | | |
| Cottonelle ® | 77.911 | 43.881 | −14.171 | 46.112 | 342.103 |
| Commercial | | | | | |
| (9.4 kg/mt | | | | | |
| cationic softener) | | | | | |
| Cottonelle ® | −25.383 | 56.057 | −17.227 | 57.134 | 63.902 |
| (15 kg/mt | | | | | |
| cationic softener) | | | | | |
| DSX Basesheet | −4.172 | 3.776 | 1.512 | 3.914 | 5.826 |
| (0% SILGARD) | | | | | |
| DSX Basesheet | −24.406 | 46.832 | −6.262 | 44.792 | 53.180 |
| (0.7% SILGARD) | | | | | |

TABLE 2
Calculated Data: CIELAB Color Space Data Relative
To Unstained TestSubstrate

| Sample | DL* | Da* | Db* | DC | DE*ab |
|---|---|---|---|---|---|
| Cottonelle ® | −7.691 | 17.423 | −6.326 | 17.231 | 20.068 |
| (0 kg/mt cationic | | | | | |
| softener) | | | | | |
| Scott ® Commercial | −8.112 | 20.556 | −6.757 | 20.830 | 23.109 |
| Northern ® Commercial | −13.298 | 32.545 | −10.999 | 31.292 | 36.838 |
| Charmin ® Commercial | −19.239 | 41.456 | −14.176 | 41.764 | 47.850 |
| Cottonelle ® | −19.618 | 44.038 | −15.192 | 45.079 | 50.547 |
| Commercial | | | | | |
| (9.4 kg/mt cat ionic | | | | | |
| softener) | | | | | |
| Cottonelle ® | −25.383 | 56.057 | −17.227 | 57.134 | 63.902 |
| (15 kg/mt cationic | | | | | |
| softener) | | | | | |
| DSX Basesheet | −4.172 | 3.776 | 1.512 | 3.914 | 5.826 |
| (0% SILGARD) | | | | | |
| DSX Basesheet | −24.406 | 46.832 | −6.262 | 44.792 | 53.180 |
| (7% SILGARD) | | | | | |

Part 2:

Three separate bath tissues and two non-cellulosic, non-woven hydroentangled polyester cleaning basesheets were evaluated for their ability to remove Candida albicans from a skin tape strip. The bath tissues evaluated included: (1) Cottonelle® (0 kg/mt of cationic softener); (2) Cottonelle® Commercial (9.4 kg/mt of cationic softener); and (3) Cottonelle® (15 kg/mt of cationic softener). The cleaning basesheets included a non-cellulosic, non-woven hydroentangled polyester sheet (DSX) without the addition of any SILGARD and a non-cellulosic, non-woven hydroentangled polyester sheet (DSX) with the addition of 0.7% (by weight) SILGARD.

Candida albicans (ATCC 10231) was obtained from the American Type Culture Collection (ATCC) (Rockville, Md.) and was subcultured for two days prior to experimentation onto a Sabourands medium fortified with glucose (SAB-Dex) agar plate (Becton Dickinson, Cockeysville, Md.) overnight at 37° C. The following day, 2-3 isolated Candida albicans colonies were inoculated into SAB-Dex Broth (20 mL) and incubated for 18 hours at 32° C. while shaking at 220 rpm. The resulting broth culture was diluted to 1×10⁵ CFU/mL with phosphate buffer (pH=7.2) (VWR Industries, Batavia Ill.).

The following procedure was utilized to determine the ability of each bath tissue to remove Candida albicans from skin tape strips. Skin tape strips were made by pulling D-Squame skin sampling disks (CuDerm Corporation, Dallas, Tex.) four times from adjacent adult volar forearm sites. The skin tape strips were then placed into deep six-well plates (Becton Dickinson, Franklin Lakes, N.J.). The skin tape strips were then blocked with 2 mL of 5% Bovine Serum Albumin (BSA) (Sigma, St. Louis, Mo.) in Phosphate Buffer Solution (150 mM NaCl, 50 mM Potassium Phosphate at a pH=7.4) for 60 minutes and shaken at 220 rpm at 33° C. After shaking, each well's fluid was removed and lmL of 10⁵ CFU/mL Candida albicans was added to each tape strip. After the addition of the Candida albicans, 1 mL of Trypticase Soy Broth (Difco Labs, Detroit, Mich.) was added to each skin tape strip and the plates incubated at 33° C. while shaking at 220 rpm for 60 minutes. After shaking, the fluid was aspirated away and the skin tape strips were washed 3 times with 3 mL Tris-Buffered Saline (50 mM Tris (base), 150 mM NaCl at a pH=7.5). After washing, each skin tape strip was placed in a new 6-well plate and 0.5 mL of Tris Buffered Saline was added to each well.

After the skin tape strips were prepared, blotters, for the determination of the affinity of each bath tissue for the Candida albicans, were prepared. Blotters were prepared by placing a new D-Squame skin sampling disc over the end of a 15×45 mm borosilicate glass open top screw cap vial (Kimble Glass Inc, VWR, Chicago, Ill.) and placing the bath tissue over the sampling disc and securing it with tape. Three separate blotters were prepared, one for each test substrate. No blotter was utilized on the control skin tape strip.

Each blotter was placed over a designated skin tape strip for 3 minutes. At the beginning of the three minute period, a firm push was exerted on the bottom side of the blotter for about 1 second. After the three minutes, each blotter was removed and discarded. The fluid remaining in each well was aspirated off and each skin tape strip was washed 3 times with 3 mL of Tris Buffered Phosphate. After this washing, each skin tape strip was ready for analysis for the removal of Candida albicans.

Each skin tape strip, including the control, was fixed by adding 2 mL of 2.5% Glutaraldehyde to each well and allowing it to stand for 10 minutes. Then each skin tape strip was then washed 3 times with 3 mL of deionized water and then 1.0 mL of 1N sodium hydroxide was added to each skin tape strip. Excess sodium hydroxide was aspirated off the tape strip to produce a moist skin tape strip. Each skin tape strip was then stained by adding 0.5 mL Calcofluor White (Difco, Ann Arbor, Mich.) to the wells for about 5 minutes after which the skin tape strips were washed 3 times with 3 mL deionized water.

Once the skin tape strips were air-dried the Candida albicans cells were enumerated visually with a fluorescent microscope. The skin tape strips were placed with the white crescent label near the bottom edge onto a microscope slide perpendicular to the microscope objective. A 20× objective was employed so that the field of view dissected the skin tape strip in the middle. Only the cells in this middle field of view (an area of about 2×10⁷ micrometers²) were counted. The field of view was about 5% of the total tape strip. The percent removal of Candida albicans from the skin tape strips was calculated according to the following formula:

[(Control # of Cells−Sample # of Cells)/Control # of Cells]×100

Approximately 5000-10,000 Candida albicans cells bound to a 22 mm diameter D-Squame skin tape strip under the conditions of this experiment.

Table 3 shows the results of Part 2 of this Example:

TABLE 3

| | | Cleaning Efficacy |
|---|---|---|
| Test Bath Tissue | Positive Charge Index | (% Removal) |
| Cottonelle ® (0 kg/mt | 20.1 | 35 |
| cationic softener) | | |
| Cottonelle ® Commercial | 50.5 | 62 |
| (9.4 kg/mt cationic | | |
| softener) | | |
| Cottonelle ® (15 kg/mt | 63.9 | 81 |
| cationic softener) | | |
| DSX Sheet (0% SILGARD) | 5.8 | 48.8 |
| DSX Sheet (0.7% | 53.2 | 77.2 |
| SILGARD) | | |

As the cleaning efficacy data indicate, as the Positive Charge Index of the test substrate increases, its ability to remove Candida albicans from the skin tape also increases leading to a higher percentage removal.

In view of the above, it will be seen that the several objects of the invention are achieved. As various changes could be made in the above-described wound management products without departing from the scope of the invention, it is intended that all matter contained in the above description be interpreted as illustrative and not in a limiting sense.

What is claimed is:

1. A wound management product comprising a substrate carrying a cationic compound capable of binding microbes located on the surface of a wound, said cationic compound having an effective charge density of from about 0.1 microequivalents/g to about 8000 microequivalents/g and a positive charge imparting a Positive Charge Index of at least about 52 to said product.

2. The product as set forth in claim 1 wherein the cationic compound is selected from the group consisting of Bufloc 535, Nalco 7607, Reten 201, Cypro 515, Bufloc 5554, Busperse 5030, cationic polymers, inorganic cationic species, biological cationic polymers, modified chitosan, octadecyldimethyltrimethoxylsilpropylammonium chloride, octadecyldimethoxylsilpropylammonium chloride, polyacrylamides, diallydimethylammonium chloride, dicyandiamideformaldehyde, epichlorohydrinamine, cationic liposomes, modified starch, l-methyl-2-Noroleyl-3-oleyl-amidoethyl imidazoline methylsulfate, l-ethyl-2-Noroleyl-3-oleyl-amidoethyl imidazoline ethylsulfate, trimethylsilylmodimethicone, amodimethicone, polyquaternium-2, polyquaternium-4, polyquaternium-5, polyquaternium-7, polyquaternium-8, polyquaternium-9, polyquaternium-10, polyquaternium-ll, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-32, polyquaternium-33, polyquaternium-34, polyquaternium-35, polyquaternium-36, polyquaternium-37, polyquaternium-39, polysilicone-1, polysilicone-2, and mixtures and combinations thereof.

3. The product as set forth in claim 1 wherein the cationic compound is selected from the group consisting of quaternary compounds, polyelectrolytes, octadecyldimethoxylsilpropylammonium chloride, 1-methyl-2-Noroleyl-3-oleyl-amidoethyl imidazoline methylsulfate, and 1-ethyl-2-Noroleyl-3-oleyl-amidoethyl imidazoline ethylsulfate.

4. The product as set forth in claim 1 wherein the substrate is selected from the group consisting of coform materials, woven webs, non-woven webs, spunbonded fabrics, meltblown fabrics, wet-laid fabrics, needle punched webs, cellulosic material, and mixtures and combinations thereof.

5. The product as set forth in claim 4 wherein the product is selected from the group consisting of wraps, wipes, gauzes, gauze wraps, bandages, and dressings.

6. The product as set forth in claim 1 wherein the substrate comprises from about 0.1% (by weight of the substrate) to about 25% (by weight of the substrate) of the cationic compound.

7. The product as set forth in claim 1 wherein the substrate comprises from about 0.1% (by weight of the substrate) to about 10% (by weight of the substrate) of the cationic compound.

8. The product as set forth in claim 1 wherein the product has a Positive Charge Index of at least about 55.

9. The product as set forth in claim 1 wherein the product has a Positive Charge Index of at least about 60.

10. The product as set forth in claim 1 wherein the Positive Charge Index of the product is determined by utilizing an anionic dye binding assay.

11. The product as set forth in claim 1 wherein the Positive Charge Index of the product is determined by utilizing an anionic dye binding assay selected from the group consisting of an Eosin Y anionic dye binding assay and an Eosin B anionic dye binding assay.

12. The product as set forth in claim 1 wherein the Positive Charge Index of the product is determined by utilizing an Eosin Y anionic dye binding assay.

13. A wound management product comprising a substrate carrying a cationic compound capable of binding microbes located on the surface of a wound, said cationic compound having an effective charge density of from about 500 microequivalents/g to about 8000 microequivalents/g and a positive charge imparting a Positive Charge Index of at least about 52 to said product.

14. The product as set forth in claim 13 wherein the cationic compound is selected from the group consisting of Bufloc 535, Nalco 7607, Reten 201, Cypro 515, Bufloc 5554, Busperse 5030, cationic polymers, inorganic cationic species, biological cationic polymers, modified chitosan, octadecyldimethyltrimethoxylsilpropylammonium chloride, octadecyldimethoxylsilpropylammonium chloride, polyacrylamides, diallydimethylammonium chloride, dicyandiamideformaldehyde, epichlorohydrinamine, cationic liposomes, modified starch, 1-methyl-2-Noroleyl-3-oleyl-amidoethyl imidazoline methylsulfate, 1-ethyl-2-Noroleyl-3-oleyl-amidoethyl imidazoline ethylsulfate, trimethylsilylmodimethicone, amodimethicone, polyquaternium-2, polyquaternium-4, polyquaternium-5, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-32, polyquaternium-33, polyquaternium-34, polyquaternium-35, polyquaternium-36, polyquaternium-37, polyquaternium-39, polysilicone-1, polysilicone-2, and mixtures and combinations thereof.

15. The product as set forth in claim 13 wherein the cationic compound is selected from the group consisting of quaternary compounds, polyelectrolytes, octadecyldimethoxylsilpropylammonium chloride, 1-methyl-2-Noroleyl-3-oleyl-amidoethyl imidazoline methylsulfate, and 1-ethyl-2-Noroleyl-3-oleyl-amidoethyl imidazoline ethylsulfate.

16. The product as set forth in claim 13 wherein the substrate is selected from the group consisting of coform materials, woven webs, non-woven webs, spunbonded fabrics, meltblown fabrics, wet-laid fabrics, needle punched webs, cellulosic material, and mixtures and combinations thereof.

17. The product as set forth in claim 16 wherein the product is selected from the group consisting of wraps, wipes, gauzes, gauze wraps, bandages, and dressings.

18. The product as set forth in claim 13 wherein the substrate comprises from about 0.1% (by weight of the substrate) to about 25% (by weight of the substrate) of the cationic compound.

19. The product as set forth in claim 13 wherein the substrate comprises from about 0.1% (by weight of the substrate) to about 10% (by weight of the substrate) of the cationic compound.

20. The product as set forth in claim 13 wherein the product has a Positive Charge Index of at least about 55.

21. The product as set forth in claim 13 wherein the product has a Positive Charge Index of at least about 60.

22. The product as set forth in claim 13 wherein the Positive Charge Index of the product is determined by utilizing an anionic dye binding assay.

23. The product as set forth in claim 13 wherein the Positive Charge Index of the product is determined by utilizing an anionic dye binding assay selected from the group consisting of an Eosin Y anionic dye binding assay and an Eosin B anionic dye binding assay.

24. The product as set forth in claim 13 wherein the Positive Charge Index of the product is determined by utilizing an Eosin Y anionic dye binding assay.

25. A wound management product comprising a substrate carrying a cationic compound capable of binding microbes located on the surface of a wound, said cationic compound having an effective charge density of from about 1000 microequivalents/9 to about 8000 microequivalents/g and a positive charge imparting a Positive Charge Index of at least about 52 to said product.

26. The product as set forth in claim 25 wherein the cationic compound is selected from the group consisting of Bufloc 535, Nalco 7607, Reten 201, Cypro 515, Bufloc 5554, Busperse 5030, cationic polymers, inorganic cationic species, biological cationic polymers, modified chitosan, octadecyldimethyltrimethoxylsilpropylammonium chloride, octadecyldimethoxylsilpropylammonium chloride, polyacrylamides, diallydimethylammonium chloride, dicyandiamideformaldehyde, epichlorohydrinamine, cationic liposomes, modified starch, 1-methyl-2-Noroleyl-3-oleyl-amidoethyl imidazoline methylsulfate, 1-ethyl-2-Noroleyl-3-oleyl-amidoethyl imidazoline ethylsulfate, trimethylsilylmodimethicone, amodimethicone, polyquaternium-2, polyquaternium-4, polyquaternium-5, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-32, polyquaternium-33, polyquaternium-34, polyquaternium-35, polyquaternium-36, polyquaternium-37, polyquaternium-39, polysilicone-1, polysilicone-2, and mixtures and combinations thereof.

27. The product as set forth in claim 25 wherein the cationic compound is selected from the group consisting of quaternary compounds, polyelectrolytes, octadecyldimethoxylsilpropylammonium chloride, 1-methyl-2-Noroleyl-3-oleyl-amidoethyl imidazoline methylsulfate, and 1-ethyl-2-Noroleyl-3-oleyl-amidoethyl imidazoline ethylsulfate.

28. The product as set forth in claim 25 wherein the substrate is selected from the group consisting of coform materials, woven webs, non-woven webs, spunbonded fabrics, meltblown fabrics, wet-laid fabrics, needle punched webs, cellulosic material, and mixtures and combinations thereof.

29. The product as set forth in claim 27 wherein the product is selected from the group consisting of wraps, wipes, gauzes, gauze wraps, bandages, and dressings.

30. The product as set forth in claim 25 wherein the substrate comprises from about 0.1% (by weight of the substrate) to about 25% (by weight of the substrate) of the cationic compound.

31. The product as set forth in claim 25 wherein the substrate comprises from about 0.1% (by weight of the substrate) to about 10% (by weight of the substrate) of the cationic compound.

32. The product as set forth in claim 25 wherein the product has a Positive Charge Index of at least about 55.

33. The product as set forth in claim 25 wherein the product has a Positive Charge Index of at least about 60.

34. The product as set forth in claim 25 wherein the Positive Charge Index of the product is determined by utilizing an anionic dye binding assay.

35. The product as set forth in claim 25 wherein the Positive Charge Index of the product is determined by utilizing an anionic dye binding assay selected from the group consisting of an Eosin Y anionic dye binding assay and an Eosin B anionic dye binding assay.

36. The product as set forth in claim 25 wherein the Positive Charge Index of the product is determined by utilizing an Eosin Y anionic dye binding assay.

37. A wound management product comprising a woven web material carrying a cationic compound capable of binding microbes located on the surface of a wound, said cationic compound having an effective charge density of from about 0.1 microequivalents/g to about 8000 microequivalents/g and a positive charge imparting a Positive Charge Index of at least about 52 to said product.

38. The product as set forth in claim 37 wherein the product has a Positive Charge Index of at least about 60.

39. The product as set forth in claim 37 wherein the cationic compound is selected from the group consisting of Bufloc 535, Nalco 7607, Reten 201, Cypro 515, Bufloc 5554, Busperse 5030, cationic polymers, inorganic cationic species, biological cationic polymers, modified chitosan, octadecyldimethyltrimethoxylsilpropylammonium chloride, octadecyldimethoxylsilpropylammonium chloride, polyacrylamides, diallydimethylammonium chloride, dicyandiamideformaldehyde, epichlorohydrinamine, cationic liposomes, modified starch, 1-methyl-2-Noroleyl-3-oleyl-amidoethyl imidazoline methylsulfate, 1-ethyl-2-Noroleyl-3-oleyl-amidoethyl imidazoline ethylsulfate, trimethylsilylmodimethicone, amodimethicone, polyquaternium-2, polyquaternium-4, polyquaternium-5, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-32, polyquaternium-33, polyquaternium-34, polyquaternium-35, polyquaternium-36, polyquaternium-37, polyquaternium-39, polysilicone-1, polysilicone-2, and mixtures and combinations thereof.

40. The product as set forth in claim 37 wherein the cationic compound is selected from the group consisting of quaternary compounds, polyelectrolytes, octadecyldimethoxylsilpropylammonium chloride, 1-methyl-2-Noroleyl-3-oleyl-amidoethyl imidazoline methylsulfate, and 1-ethyl-2-Noroleyl-3-oleyl-amidoethyl imidazoline ethylsulfate.

41. A wound management product comprising a non-woven web material carrying a cationic compound capable of binding microbes located on the surface of a wound, said cationic compound having an effective charge density of from about 0.1 microequivalents/g to about 8000 microequivalents/g and a positive charge imparting a Positive Charge Index of at least about 52 to said product.

42. The product as set forth in claim 41 wherein the product has a Positive Charge Index of at least about 60.

43. The product as set forth in claim 41 wherein the cationic compound is selected from the group consisting of Bufloc 535, Nalco 7607, Reten 201, Cypro 515, Bufloc 5554, Busperse 5030, cationic polymers, inorganic cationic species, biological cationic polymers, modified chitosan, octadecyldimethyltrimethoxylsilpropylammonium chloride, octadecyldimethoxylsilpropylammonium chloride, polyacrylamides, diallydimethylammonium chloride, dicyandiamideformaldehyde, epichlorohydrinamine, cationic liposomes, modified starch, 1-methyl-2-Noroleyl-3-oleyl-amidoethyl imidazoline methylsulfate, 1-ethyl-2-Noroleyl-3-oleyl-amidoethyl imidazoline ethylsulfate, trimethylsilylmodimethicone, amodimethicone, polyquaternium-2, polyquaternium-4, polyquaternium-5, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-32, polyquaternium-33, polyquaternium-34, polyquaternium-35, polyquaternium-36, polyquaternium-37, polyquaternium-39, polysilicone-1, polysilicone-2, and mixtures and combinations thereof.

44. The product as set forth in claim 41 wherein the cationic compound is selected from the group consisting of quaternary compounds, polyelectrolytes, octadecyldimethoxylsilpropylammonium chloride, 1-methyl-2-Noroleyl-3-oleyl-amidoethyl imidazoline methylsulfate, and 1-ethyl-2-Noroleyl-3-oleyl-amidoethyl imidazoline ethylsulfate.
